(19) United States
(12) Patent Application Publication
(10) Pub. No.: US 2012/0116297 A1
(43) Pub. Date: May 10, 2012

(54) Medication Application Device for Use in Body Cavities

(76) Inventor: Cynthia Sheffield, Mt. Gilead, NC (US)

(21) Appl. No.: 13/289,133

(22) Filed: Nov. 4, 2011

Related U.S. Application Data
(60) Provisional application No. 61/411,260, filed on Nov. 8, 2010.

(51) Int. Cl. A61M 5/00

(52) U.S. Cl. 604/60

(57) ABSTRACT

A pill delivery device is provided for inserting pills into enclosed regions within human body cavities, comprising an arcuate, rigid applicator tube, a plunger and a flexible pill-retaining sleeve. The pill-retaining sleeve is secured to a lower end of the applicator tube and has an aperture disposed at a lower end of the sleeve. The aperture prevents pills from falling from the sleeve; however the sleeve is flexible to permit distortion of the aperture, thereby allowing pills to be pushed therethrough. The plunger has a handle disposed at a top end and a working end in the shape of a ball, disposed at its lower end. In use, a pill is inserted into the top end of the tube. The device is then inserted into a body cavity and the plunger is utilized to force medication through the retaining sleeve and into the body cavity.

### CROSS REFERENCE TO RELATED APPLICATION

This application claims the benefit of U.S. Provisional Application No. 61/411,260 filed on Nov. 8, 2010, entitled “Intracervical Cytotec Applicator.”

### BACKGROUND OF THE INVENTION

1. Field of the Invention

The present invention relates to a pill delivery device. More specifically, the present invention relates to a device for administering a plurality of pills into a hard to reach regions of a human body cavity.

When a woman is in labor, it is essential that her cervix properly expands to permit the infant to pass through and into the vaginal cavity. To facilitate proper dilation of the cervix, healthcare providers will often manually insert dissolvable pills into the entrance of the cervical canal. These pills are absorbed by the surrounding body tissue and result in the relaxation of the cervical opening and dilation thereof. This procedure is problematic because the pills can easily become dislodged before they are fully absorbed or incorrectly placed for proper function. If the pills become dislodged after a healthcare provider has removed his or her hand from the vaginal cavity, the displacement of medicine may go unnoticed for a substantial time. This could result in extended labor and longer periods of discomfort for the mother. A pill delivery device is needed that is easily insertable into a body cavity and is capable of placement in enclosed or otherwise difficult to reach areas of the body. Such a device would facilitate the delivery of pills into the cervical canal with minimal discomfort and would facilitate easy replacement of pills that become dislodged. The device can further be used in other healthcare or veterinary situations in which pill delivery is required within a body cavity wherein access to is limited.

2. Description of the Prior Art

The prior art contains a variety of pill delivery devices for providing easy application of medicine in pill form to various cavities of a human body. These devices have familiar design and structural elements for the purposes of administering pills to patients; however they are not adapted for the task of administering multiple pills into oddly shaped cavities of the human body.

Propp, U.S. Pat. No. 6,123,683 discloses a pill delivery device for facilitating the insertion of medication into a body cavity. The device comprises a flexible conduit having a working end and a delivery end, a plunging wire secured within said flexible conduit and a flexible sleeve disposed at said delivery end. The plunging wire has a handle disposed at one end and an opposing end that is inserted through the flexible conduit. A flexible sleeve is secured to the delivery end of the flexible conduit and has an opening at the opposing end. The diameter of the opening is smaller than that of the flexible sleeve to prevent pills from falling out. A pill is inserted into the device through the small opening in the flexible sleeve by distorting said sleeve around the shape of the pill. The device is then inserted into a body cavity and the plunging wire is pressed forward such that the end of the wire presses the pill through the opening at the end of the flexible sleeve. Some procedures require the insertion of multiple medications into a body cavity. The device of Propp does not accommodate the need for storing multiple pills within the device. The present invention provides a top loaded device for the insertion of multiple pills into the conduit so the device does not have to be extracted after each pill insertion. This causes less discomfort to the user and provides for faster application of medication. Furthermore the device of Propp does not disclose a rigid conduit like that of the present invention.

Hanson, U.S. Pat. No. 4,060,083 discloses a pill insertion gun for delivering medicine to small animals. The pill gun comprises a rigid barrel, a plunging means and finger grips disposed at an end of the same. The plunging means is a thin rod inserted into the rigid barrel and having a “T” shaped handle. One end of the tube is blunt and is adapted to permit pills to be inserted therein. The end of the rigid barrel nearest to the plunging handle is also “T” shaped. A user's fingers may be placed between the plunging handle and the “T” bar of the rigid barrel to assist with retention of the pills in the barrel. The rigid barrel may then be inserted into the mouth of a small animal and the plunger depressed to deliver the pills. Hanson does not disclose a curved tube suitable for insertion into the curved cavities of a human body. Nor does Hanson disclose a flexible sleeve disposed at the working end of the barrel for assisting with retention of pills. The present invention provides for both of these elements.

Spead, U.S. Pat. No. 5,662,601 discloses a suppository applicator. The device has a hollow, cylindrical barrel and a plunger shaft. The plunger shaft is inserted through one end of the barrel and is guided therethrough to a working end of the barrel. The plunging shaft has a handle disposed at one end to allow a user to grip the device. At the end of the hollow barrel opposing the plunging shaft handle, there is a suppository shield. The suppository shield is flexible to assist with insertion of the device. Pills are loaded into the delivery end of the device, which is then inserted into a patient's anal cavity and the plunger is then depressed to deliver pills. The barrel of Spead is short and is not arcuate like the long, curved tube of the present invention. This shortened, straight barrel makes Spead incompatible with the intended use of the present invention.

DiPiano et al, U.S. Pat. No. 7,591,808 discloses a vaginal or rectal medicine applicator. The device comprises an applicator barrel, a plunger, a plunger tip and an applicator cap. The applicator is similar in appearance and function to a syringe. The applicator barrel is filled with liquid medicine by submerging the tip of the applicator into the medicine and sliding the plunger into a back position. The applicator is then removed from the liquid and the cap placed on the end of the applicator to seal the liquid in the barrel. To use the device the cap is removed, the applicator is inserted into a cavity and the plunger is depressed. The size of the applicator barrel makes it unsuitable for retention and delivery of pills. DiPiano does not disclose a long, arcuate tube that may be inserted into the cervix of a patient.

Roper et al, U.S. Pat. No. 5,681,279 discloses a pill dispensing syringe comprising a cylindrical outer housing, a water injection cylinder slidably mounted within the interior bore of the outer housing, a plunger slidably mounted within the interior bore of the water injection cylinder and a cylindrical pill dispensing sleeve secured to the delivery end of the housing. The dispensing sleeve is adapted to retain a pill. The water injection cylinder holds water while the plunger is slid into a “back” position. When the plunger is depressed the end of the plunger forces the pill forward for delivery into the mouth of a patient, water is also dispensed into the mouth, to assist in the swallowing of said pill. This device does not permit the top loading of multiple pills unlike the present invention. Additionally Rpper does not disclose an arcuate, rigid tube suitable for insertion into curved cavities of the human body. The size and shape of the Roper device make it unsuitable for the insertion of pills into a cervical canal.

The devices disclosed by the prior art do not address the need for delivery of multiple pills into hard to reach portions of human body cavities. The current invention relates to a device for inserting at least one pill into a cervical canal. It substantially diverges in structural elements from the prior art; consequently it is clear that there is a need in the art for an improvement to the current pill delivery devices. In this regard the instant invention substantially fulfills these needs.

### SUMMARY OF THE INVENTION

In view of the foregoing disadvantages inherent in the known types of pill delivery devices now present in the prior art, the present invention provides a new, multiple pill retention capability wherein the same can be utilized for providing convenience for the user when delivering multiple pills to a hard to reach portion of a human body cavity.

The present invention comprises an applicator tube, a plunger and a flexible pill retainer. The applicator tube is rigid and curved to facilitate easy insertion and removal of the tube from oddly shaped areas of a body cavity. The plunger is a rod with a handle disposed at one end. This rod is inserted into the applicator tube and can slide therein. At an opposing end of the applicator tube, the flexible pill retainer sleeve is secured. The flexible retainer has a small aperture disposed at an end opposing the applicator tube. The aperture is sized to prevent pills from falling out of the retaining sleeve, but allow said pills to be pushed through the aperture by the plunger.

The present invention is best suited for use in obstetrical medicine; however it may be utilized for a variety of uses, including anal and oral delivery of pills. The vaginal body cavity is curved upward from the thigh region, making it difficult for straight devices to be effectively inserted. The curved, rigid nature of the applicator tube is ideal for insertion of the device through the vagina and to the cervix. To deliver pills into this area, the healthcare professional inserts the medicine into the top end of the applicator tube and shakes the tube gently to assist the pills with moving through the tube and into the pill-retaining sleeve. The plunger is then inserted into the top end of the applicator tube and slid therethrough until gentle contact is made with the pills. The caregiver then guides the device into the vagina with his or her hand and moves the end of the pill-retaining sleeve into position against the opening of the cervix. The plunger is then depressed, resulting in the delivery of a desired dose of pill-form medicine into the cervical canal where the medication will remain after removal of the present invention.

It is therefore an object of the present invention to provide a new and improved pill delivery device having all of the advantages of the prior art and none of the disadvantages.

Another object of the present invention is to provide a new and improved pill delivery device having a curved, rigid applicator tube suitable for easy insertion into the vaginal cavity of a woman.

Still another object of the present invention is to provide a new and improved pill delivery device having a lengthy, curved applicator tube for facilitating the delivery of medicine into hard to reach areas of a human body cavity.

Another object of the present invention is to provide a new and improved pill delivery device having a top-loaded hollow applicator tube providing delivery of multiple pills.

Yet another object of the present invention is to provide a new and improved pill delivery device having durable and resilient construction.

Other objects, features and advantages of the present invention will become apparent from the following detailed description taken in conjunction with the accompanying drawings.

### BRIEF DESCRIPTIONS OF THE DRAWINGS

The above invention will be better understood and the objects set forth above as well as other objects not stated above will become more apparent after a study of the following detailed description thereof. Such description makes use of the annexed drawings wherein like numeral references are utilized throughout.

FIG. 1 shows a perspective view of the present pill delivery device while pills are loaded into the applicator tube.

FIG. 2 shows a perspective view of the present pill delivery device as the plunger is inserted into the applicator tube.

FIG. 3 shows a perspective view of the present pill delivery device as inserted into the vaginal cavity of a pregnant woman.

### DETAILED DESCRIPTION OF THE INVENTION

Reference is made herein to the attached drawings. Like reference numerals are used throughout the drawings to depict like or similar elements of the pill delivery device. For the purposes of presenting a brief and clear description of the present invention, the preferred embodiment will be discussed as used for insertion of multiple pills into hard to reach portions of a human body cavity. This is for representative purposes only and should not be considered to be limiting in any respect.

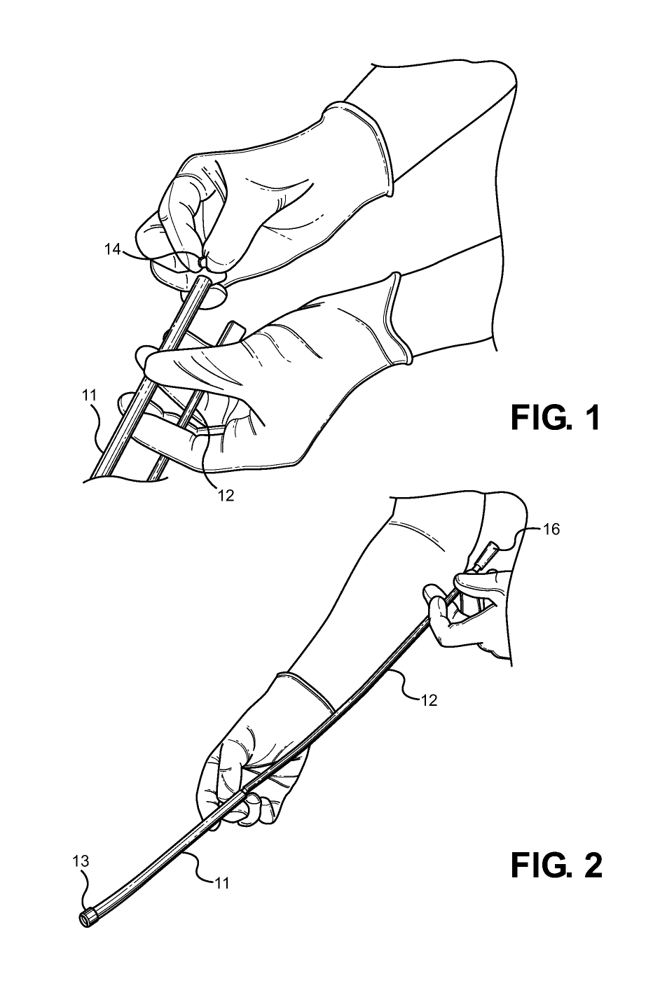

Referring now to FIG. 1, there is shown a pill delivery device. Pills 14 are first inserted into a top end of the applicator tube 11. The applicator tube comprises a long, hollow tube. It is constructed of a rigid, sanitizable material such as plastic or metallic material. To facilitate easy insertion of the device into curved portions of a body cavity, the applicator tube has an arcuate shape. The curve of the tube should be gradual; however a variety of curvature options are contemplated. Gentle curvature of the applicator tube makes the device ideal for insertion into areas such as vaginal or rectal cavities, along within the throat region. In a preferred embodiment, the tube is cylindrical and is adapted to receive a variety of pill sizes. Because pills 14 are loaded into the top of the applicator tube 11, multiple pills may be inserted at the same time. This functionality permits the delivery of more than one pill into a body cavity at one time, thus relieving the user of discomfort by reducing the need for multiple insertions of the device. As shown in the figure, the tube 11 may be filled with pills 14 while simultaneously holding the plunger 12, improving speed of the overall operation and eliminating any need for a sterile support surfaces prior to use.

Referring now to FIG. 2, there is shown a pill delivery device as described by the present invention while a plunger 12 is being inserted into the hollow cavity of the applicator tube 11. The plunger 12 has a handle 16 disposed at its proximal end to assist a user with grip thereof. This handle may have a variety of shapes, including a circle, a “T” bar or the like. At the distal end of the plunger is the working end. In the preferred embodiment, the working end has a ball secured thereto. The ball has a diameter slightly less than that of the applicator tube interior diameter so that the ball may slide freely within the tube. When the ball comes into contact with pills inserted into the tube, the pills are forced through the length of the tube by depressing the plunger handle into the tube. In alternative embodiments, the working end may be disc shaped, conical, or employ a blunt terminus.

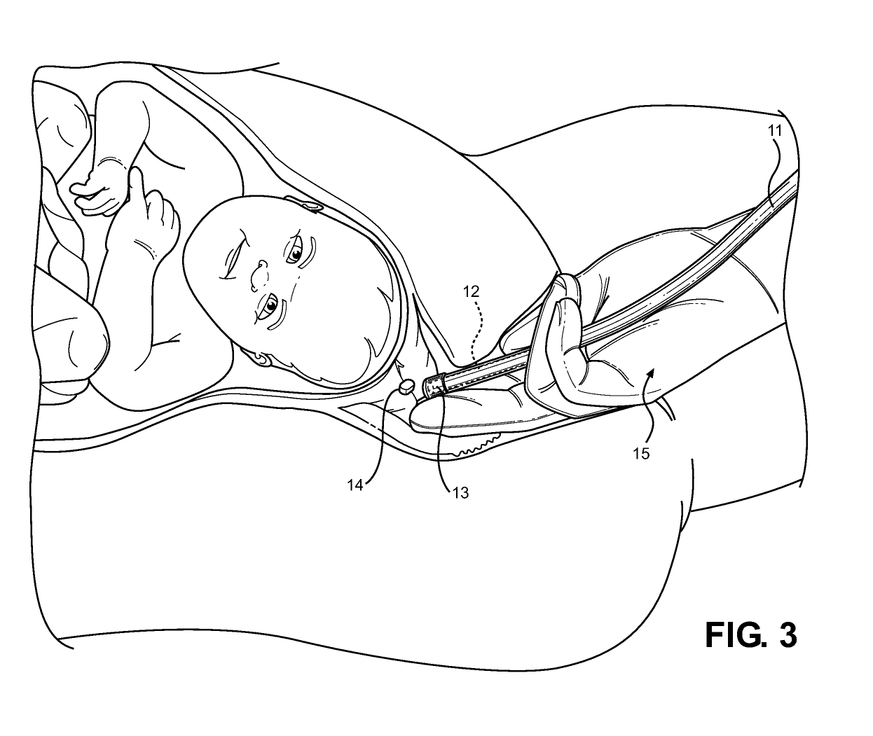

Referring now to FIG. 3 there is shown a pill delivery device in use. After pills 14 and the plunger 12 have been inserted into the applicator tube 11, the device is gently inserted into a body cavity. A flexible pill-retaining sleeve 13 is secured to the working lower end of the applicator tube 11. This pill-retaining sleeve has a small aperture disposed at its lower end. The aperture is smaller than the diameter of the applicator tube 11 and the retaining sleeve 13 to prevent pills from falling out prior to delivery. However, the retaining sleeve 13 is constructed of a flexible membrane that permits the aperture to distort and stretch. When the plunger 12 is fully depressed, pills 14 are pushed through the aperture and into the target delivery site. The flexible construction of the sleeve 13 also facilitates comfortable insertion of the device into a body cavity. The risk of injury to interior parts of a body cavity is reduced due to the lack of a rigid, blunt tip that could bruise or scrape sensitive tissue. Additionally, the ability of the device to reach curved portions of a body cavity increases the likelihood of successful placement of delivered pills, thereby increasing effectiveness of treatments.

In use an individual inserts at least one pill into the applicator tube and shakes the tube gently to help the pill travel downward through he applicator tube. The user then holds the applicator tube and inserts the plunger into a top end of the tube, which is used to force the pills towards the working end of the tube. The plunger is pushed downward through the tube until the working end abuts the medication resting in a working end of the applicator tube. A user may then insert one hand 15 into the entrance to the vaginal cavity and use the other hand to slide the device into the vagina using the inserted hand as a guide. The device is positioned so that the aperture of the flexible pill-retaining sleeve is gently inserted into the entrance of the cervical canal. The plunger is then depressed to deliver the pills into the cervical canal. A user may then slide the device out of the vaginal cavity and remove the inserted hand. Similar steps may be used to execute delivery of medication into oral and anal cavities.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A pill delivery device, comprising:
a hollow, arcuate applicator tube;
a flexible, pill-retaining sleeve secured to a working end of said applicator tube and having an aperture;
a plunger having a handle disposed at a first end and a working end disposed in opposition of said handle;
said plunger adapted to fit through said applicator tube and force pills therethrough.

2. The device of claim 1, wherein said applicator tube is rigid.

3. The device of claim 1, wherein said pill-retaining sleeve aperture further comprises a smaller diameter than said applicator tube interior diameter to retain pills within said tube until said plunger forces pills through said aperture.

4. The device of claim 1, wherein said working end of said plunger is ball-shaped.

5. The device of claim 1, wherein said working end of said plunger is disc shaped.

6. The device of claim 1, wherein said working end of said plunger is conical.

7. The device of claim 1, wherein said plunger handle is spherical.

8. The device of claim 1, wherein said plunger handle is a crossbar.

9. A pill delivery device, comprising:
a hollow, rigid, arcuate applicator tube;
a flexible pill-retaining sleeve secured to a lower end of said applicator tube and having an aperture;
a plunger having a handle disposed at a first end and a ball-shaped working end disposed in opposition to said handle;
said aperture further comprising a smaller diameter than said applicator tube interior diameter to retain pills within said tube until said plunger forces pills through said aperture;
said plunger adapted to fit through said applicator tube and force pills therethrough.
